(19) United States
(12) Patent Application Publication
(10) Pub. No.: US 2025/0222110 A1
(43) Pub. Date: Jul. 10, 2025

(54) METHODS AND COMPOSITIONS FOR TREATMENT OF AGE-RELATED CATARACTS

(71) Applicant: LIVIONEX, Inc., Los Gatos, CA (US)

(72) Inventors: Rajiv BHUSHAN, Los Gatos, CA (US); Amit GOSWAMY, Los Gatos, CA (US); Jarry GIN, Sunnyvale, CA (US)

(21) Appl. No.: 19/094,981

(22) Filed: Mar. 30, 2025

Related U.S. Application Data
(60) Provisional application No. 63/679,157, filed on Aug. 4, 2024.
(60) Provisional application No. 63/700,508, filed on Sep. 27, 2024.

(51) Int. Cl. A61K 47/20; A61K 31/19; A61K 47/22; A61K 47/34; A61K 47/38; A61P 27/12

(52) U.S. Cl. CPC A61K 47/20; A61K 31/19; A61K 47/22; A61K 47/34; A61K 47/38; A61P 27/12

(57) ABSTRACT

A noninvasive ophthalmic formulation, comprising a chelator (such as EDTA and its salts), a transport enhancer (such as Methyl Sulfonyl Methane; MSM), a second permeation enhancer such as propylene glycol, a thickening agent such as hydroxyethyl cellulose (HEC) and a surfactant such as polysorbate 80, is provided. The novel combination of the ingredients unexpectedly and beneficially reduces symptoms associated with early-stage, age-related cataracts. Methods for treating cataracts with compositions of the invention and measurement of improved visual function by measuring contrast sensitivity (CS) are provided.

### CROSS-RELATION TO RELATED APPLICATIONS

This U.S. national application for patent is filed under 35 U.S.C. 111 (a) and claims priority to U.S. provisional patent application Ser. No. 63/679,157 filed Aug. 4, 2024 titled “METHODS AND COMPOSITIONS FOR TREATMENT OF AGE-RELATED CATARACTS,” and U.S. provisional patent application Ser. No. 63/700,508 filed Sep. 27, 2024, titled “METHODS AND COMPOSITIONS FOR TREATMENT OF AGE-RELATED CATARACTS,” both of which are incorporated herein by reference.

### TECHNICAL FIELD OF THE INVENTION

This invention relates generally to the treatment of ophthalmic disorders, including age-related cataracts. In particular, the invention pertains to the non-invasive treatment of conditions associated with age-related cataracts using intracellular iron and calcium as therapeutic targets. In particular, the invention relates to antimicrobial compositions containing one or more transport enhancers, a chelating agent, a charge-masking agent, and optionally, a detergent. In an exemplary embodiment, it relates to topical application compositions containing MSM, chelators, propylene glycol (PG), hydroxyethyl cellulose (HEC), and Tween®80.

### BACKGROUND OF THE INVENTION

Progressive, age-related changes of the eye, including regular and pathological changes, are an inevitable part of life in humans and other mammals. Many of these changes seriously affect both the function and the cosmetic appearance of the eyes. These changes include the development of cataracts; hardening, opacification, reduction of pliability, and yellowing of the lens; yellowing and opacification of the cornea; presbyopia; clogging of the trabecula, leading to intraocular pressure build-up and glaucoma; increased floaters in the vitreous humor; stiffening and reduction of the dilation range of the iris; age-related macular degeneration (AMD); formation of atherosclerotic deposits in retinal arteries; dry eye syndrome; and decreased sensitivity and light level adaptation ability of the rods and cones of the retina. Age-related vision deterioration includes loss in visual acuity, visual contrast, color and depth perception, lens accommodation, light sensitivity, and dark adaptation. Age-related changes also include changes in the color appearance of the iris and the formation of arcus senilis.

Age-related cataracts, also known as senile cataracts, is a common eye condition characterized by the clouding and thickening of the natural lens in the eye, leading to decreased vision. This condition typically develops because of aging and is a leading cause of vision impairment and blindness in older adults. Effective non-invasive therapy to treat visual function decline caused by pre-surgical cataracts remains a significant unmet medical need in the US and the rest of the world. It is estimated that over 80 million people worldwide have moderate and severe vision impairment caused by cataracts. (GBD 2019 Blindness and Vision Impairment Collaborators. Causes of blindness and vision impairment in 2020 and trends over 30 years, and prevalence of avoidable blindness in relation to VISION 2020: the Right to Sight: an analysis for the Global Burden of Disease Study. Lancet Glob Health 2021; 9: e130-43; published online Dec. 1, 2020. https://doi.org/10.1016/S2214-109X(20)30489-7).

Disturbances in calcium homeostasis are associated with various forms of cataracts. Ca²⁺-mediated disintegrative globulization of the fiber cells may be responsible for forming light-scattering centers during cataractogenesis. Age-related cataract is a multifactorial degenerative disorder in which excess free iron catalyzes the generation of reactive oxygen species (ROS), and elevated intracellular calcium initiates the inflammatory cascade and binds damaged proteins as light-scattering particles in the lens. (Wang L, Bhatnagar A, Ansari NH, Dhir P, Srivastava SK. Mechanism of calcium-induced disintegrative globulization of rat lens fiber cells. Invest Ophthalmol Vis Sci. 1996; 37(5):915-922).

While metal chelation is a well-understood scientific process, its therapeutic effectiveness in clinical settings has been limited, primarily due to the inability of chelator molecules to permeate biological tissues and cell membranes. Preclinical (in vivo) pharmacokinetic findings demonstrated that C-KAD greatly enhanced the delivery of EDTA into various intraocular tissues, including the lens. (Zhang M, Wong IG, Gin JB, Ansari NH. Assessment of methylsulfonylmethane as a permeability enhancer for regional EDTA chelation therapy. Drug Deliv. 2009; 16(5):243-248. doi:10.1080/10717540902896362) Another preclinical (in vivo and in vitro) study has also shown that C-KAD reduced oxidation-induced lens opacification and cellular toxicity, as well as the inflammatory cascade associated with oxidative stress and cataract formation. (Zhang M, Shoeb M, Liu P, et al. Topical metal chelation therapy ameliorates oxidation-induced toxicity in diabetic cataracts. J. Toxicol. Environ. Health A. 2011; 74(6):380-391).

Currently, there are no Food and Drug Administration (FDA) approved pharmacological therapies for age-related cataracts, and cataract surgery remains the only safe and effective treatment option available. Although cataract surgery has become common in many parts of the world, the number of individuals with vision loss associated with cataracts has been increasing due to an aging population in many developed countries. (Bourne RRA, Flaxman SR, Braithwaite T, et al. Magnitude, temporal trends, and projections of the global prevalence of blindness and distance and near vision impairment: a systematic review and meta-analysis. Lancet Glob Health. 2017; 5(9):e888-e897). Since the disease progression and visual function loss occur gradually over time, patients can experience and suffer visual function decline for years before cataract surgery becomes available for those with late-stage cataracts.

The inventors of the present application have previously disclosed formulations comprising chelators and permeation enhancers for the treatment of ophthalmic disorders. International application no. PCT/US2006/027686 (WO2007011875) disclosed formulations for reducing macromolecular aggregates in the eye with formulations comprising metal chelators and charge-masking agents such as MSM and EDTA. PCT/US2006/027685 (WO2007011874) disclosed formulations comprising metal chelators and charge-masking agents such as MSM and EDTA for treating ocular conditions associated with aging. US Patent application no. PCT/US2003/041141 (WO 2004/058289) disclosed formulations containing a biocompatible chelating agent, an effective permeation-enhancing amount of an ophthalmic permeation enhancer such as methylsulfonylmethane (MSM), and an anti-AGE (advanced glycation end products) agent for treating adverse ocular conditions. However, these formulations were found to be insufficiently effective against age-related cataracts when measured by standard tests.

It has been hypothesized that topical chelation using C-KAD can slow down the progression and reverse cataract progression, thereby improving visual function in patients with early cataracts. (Micun Z, Falkowska M, Młynarczyk M, Kochanowicz J, Socha K, Konopińska J. Levels of Trace Elements in the Lens, Aqueous Humour, and Plasma of Cataractous Patients-A Narrative Review. Int J Environ Res Public Health. 2022; 19(16):10376. Published 2022 Aug. 20. doi:10.3390/ijerph191610376). The inventors of the instant application have identified intracellular iron and calcium as therapeutic targets for age-related cataracts.

### SUMMARY OF THE INVENTION

The present invention is based on the surprising observation that the use of formulation with Ethylenediaminetetraacetic Acid Disodium (EDTA; also referred to as C-KAD) as an active ingredient for chelation of intracellular iron and calcium is successful as a topical therapy for age-related cataracts.

The inability of chelator molecules to permeate biological tissues and cell membranes is overcome by including a plurality of permeation enhancers and detergents such as methylsulfonylmethane (MSM), propylene glycol (PG) and/or hydroxyethyl cellulose, and Tween® 80.

According to the invention, compositions contain a percentage of chelator at about 0.01% to 15% w/w, and the percentage of permeation enhancer in the composition is about 0.01%-30% w/w, respectively. Compounds of this class include but are not limited to a glycol ether, alkyl ethers of diethylene glycol, butylene glycol, dipropylene glycol, ethylene glycol, polyethylene glycol (PEG), or propylene glycol (PG) added as a second permeation enhancer at about 0.1%-10% w/w. Compositions of the invention may further comprise a thickening agent such as, by way of example, cellulosic polymers such as methylcellulose (MC), hydroxyethylcellulose (HEC), hydroxypropylcellulose (HPC), hydroxypropyl-methylcellulose (HPMC), and sodium carboxymethylcellulose (NaCMC), and other swellable hydrophilic polymers such as polyvinyl alcohol (PVA), hyaluronic acid or a salt thereof. A polysorbate detergent, such as Tween® 80, can be incorporated optionally into the compositions of the invention at 0.1%-40% w/w.

In some embodiments, the present invention relates to methods for the use of the formulations comprising a transport enhancer (such as MSM) and a chelating agent (such as EDTA) and concentrations of PG between 0.5% to 5% and an ophthalmologically acceptable inert carrier, for alleviation of adverse ophthalmic conditions caused by age-related cataracts.

The method involves administering to the subject an effective amount of a formulation composed of a therapeutically effective amount of a chelating agent and an effective transport-enhancing amount of a transport enhancer having the formula (I)

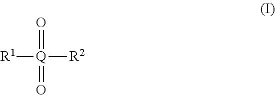

wherein R¹ and R² are independently selected from C₂-C₆ alkyl, C₁-C₆ heteroalkyl, C₆-C₁₄ aralkyl, and C₂-C₁₂ heteroaralkyl, any of which may be substituted, and Q is S or P.

A first transport-enhancing agent can be a charge-masking agent like methylsulfonylmethane (MSM; also referred to as methylsulfone, dimethylsulfone, and DMSO2); the chelating agent can be ethylene diamine tetraacetic acid (EDTA) and the like. A second permeation enhancer can be a polyoxyalkylene, which may be selected from the group consisting of: glycol ether, alkyl ethers of diethylene glycol, butylene glycol, dipropylene glycol, ethylene glycol, polyethylene glycol (PEG), propylene glycol (PG) and derivatives thereof.

An exemplary embodiment comprises MSM at 5.4% w/w; di-sodium EDTA at 2.6% w/w; propylene glycol at 5.0% w/w, hydroxyethylcellulose (HEC) at 0.3%, TWEEN®80 (polysorbate 80) at 15.0% w/w, and optionally, guar gum or hydroxypropyl guar at 0.1% w/w.

The formulation may be administered in any form suitable for ophthalmic administration, including liquid and gel-based compositions. Additionally, in a preferred embodiment, the formulation is entirely composed of components that are naturally occurring and/or as GRAS (“Generally Regarded as Safe”) by the U.S. Food and Drug Administration.

The invention also pertains to a method for preventing and treating adverse ocular conditions, including those involving oxidative and/or free radical damage in the eye, some of which are associated with forming or depositing macromolecular aggregates. The formulation contains a therapeutically effective amount of an ophthalmologically active agent, a sequestrant of metal cations, e.g., a chelating agent as described above, and a transport enhancer as also described above. These adverse ocular conditions include, for example, conditions, diseases, or disorders of the cornea, retina, lens, sclera, and anterior and posterior segments of the eye. An adverse ocular condition, as used herein, can be a “normal” condition frequently seen in aging individuals (e.g., decreased visual acuity and contrast sensitivity) or a pathologic condition that may or may not be associated with the aging process. The latter adverse ocular conditions include a wide variety of ocular disorders and diseases.

Aging-related ocular problems that can be prevented and/or treated using the present formulations include, without limitation, opacification (both corneal and lens opacification), cataract formation (including secondary cataract formation) and other problems associated with deposition of lipids, visual acuity impairment, decreased contrast sensitivity, photophobia, glare, dry eye, loss of night vision, narrowing of the pupil, presbyopia, age-related macular degeneration, elevated intraocular pressure, glaucoma, and arcus senilis. By “age-related,” it means a condition that is generally recognized as occurring far more frequently in older patients but that may and occasionally does occur in younger people. The formulations can also be used to treat ocular surface growths such as pingueculae and pterygia, which are typically caused by dust, wind, or ultraviolet light, but may also be symptoms of degenerative diseases associated with the aging eye. Another adverse condition that is generally not viewed as aging-related but which can be treated using the present formulation includes keratoconus. It should also be emphasized that the present formulation can be advantageously employed to improve visual acuity, in general, in any mammalian individual. That is, ocular administration of the formulation can improve visual acuity and contrast sensitivity as well as color and depth perception regardless of the patient's age or the presence of any adverse ocular conditions. The formulation is useful for treating adverse ophthalmic conditions in both humans and animals.

These and other aspects will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

The following drawings form part of the present specification and are included to demonstrate further certain aspects of the present disclosure, the inventions of which can be better understood by reference to one or more of these drawings in combination with the detailed description of specific embodiments presented herein.

FIG. 1 shows the area under the Log Contrast Sensitivity Function (Mean±SE in log CS). Mean area under the log contrast sensitivity function (AULCSF) under mesopic condition as a summary metric for overall contrast sensitivity function (CSF) is presented from baseline to Day 120 for C-KAD and placebo groups. AULCSF allows for quantitative comparison of overall CSFs over time and/or between groups. (ª Statistically significant with P<0.05)

FIG. 2 shows the mean Contrast Sensitivity Function. The contrast sensitivity function (CSF) is plotted as a curve with the mean log CS on the Y-axis and spatial frequencies from 1.5 to 18 cpd on the X-axis on a log scale at baseline and Day 120 for C-KAD and placebo groups. CSF presents contrast sensitivity as a function of spatial frequency.

### DETAILED DESCRIPTION OF THE INVENTION

The terms used in this specification generally have their ordinary meanings in the art, within the context of the invention, and in the specific context where each term is used. Certain terms used to describe the invention are discussed below or elsewhere in the specification to provide additional guidance to the practitioner regarding the description of the invention. Synonyms for specific terms are provided. A recital of one or more synonyms does not exclude using other synonyms. The use of examples anywhere in this specification, including examples of any terms discussed herein, is illustrative only and in no way limits the scope and meaning of the invention or any exemplified term. Likewise, the invention is not limited to the various embodiments given in this specification.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any expressly excluded limit in the stated range. Where the stated range includes one or both of the limits, ranges excluding either or both of those included limits are also included in the invention.

Various publications, patents, and published patent applications are cited throughout this application. The inventions of these publications, patents, and published patent applications referenced in this application are hereby incorporated by reference in their entireties into the present invention. Citation herein of a publication, patent, or published patent application is not an admission the publication, patent, or published patent application is applicable as prior art.

When referring to a formulation component, it is intended that the term used, e.g., “agent,” encompasses not only the specified molecular entity but also its pharmaceutically acceptable analogs, including, but not limited to, salts, esters, amides, prodrugs, conjugates, active metabolites, and other such derivatives, analogs, and related compounds.

The terms “treating” and “treatment” as used herein refer to the administration of an agent or formulation to a clinically symptomatic individual afflicted with an adverse condition, disorder, or disease to affect a reduction in severity and/or frequency of symptoms, eliminate the symptoms and/or their underlying cause, and/or facilitate improvement or remediation of damage. The terms “preventing” and “prevention” refer to administering an agent or composition to a clinically asymptomatic individual susceptible to a particular adverse condition, disorder, or disease and thus relates to the prevention of symptoms and/or their underlying cause. Unless otherwise indicated herein, either explicitly or by implication, if the term “treatment” (or “treating”) is used without reference to possible prevention, it is intended that prevention be encompassed as well, such that “a method for the treatment of gingivitis” would be interpreted as encompassing “a method for the prevention of gingivitis.”

“Optional” or “optionally present”—as in an “optional substituent” or an “optionally present additive” means that the subsequently described component (e.g., substituent or additive) may or may not be present so that the description includes instances where the component is present and instances where it is not.

By “pharmaceutically acceptable,” it means a material that is not biologically or otherwise undesirable, e.g., the material may be incorporated into a formulation of the invention without causing any undesirable biological effects or interacting in a deleterious manner with any of the other components of the dosage form formulation. However, when the term “pharmaceutically acceptable” is used to refer to a pharmaceutical excipient, it is implied that the excipient has met the required standards of toxicological and manufacturing testing and/or that it is included on the Inactive Ingredient Guide prepared by the U.S. Food and Drug Administration. As explained in further detail infra, “pharmacologically active” (or simply “active”) as in a “pharmacologically active” derivative or analog refers to a derivative or analog having the same type of pharmacological activity as the parent agent. The terms “treating” and “treatment” as used herein refer to a reduction in severity and/or frequency of symptoms, elimination of symptoms and/or underlying cause, prevention of the occurrence of symptoms and/or their underlying cause, and improvement or remediation of an undesirable condition or damage. Thus, for example, “treating” a subject involves the prevention of an adverse condition in a susceptible individual as well as the treatment of a clinically symptomatic individual by inhibiting or causing regression of the condition. The term “chelating agent” (or “active agent”) refers to any chemical compound, complex or composition that exhibits a desirable effect in the biological context, i.e., when administered to a subject or introduced into cells or tissues in vitro. The term includes pharmaceutically acceptable derivatives of those active agents specifically mentioned herein, including, but not limited to, salts, esters, amides, prodrugs, active metabolites, isomers, analogs, crystalline forms, hydrates, and the like. When the term “chelating agent” is used, or when a particular chelating agent is specifically identified, it is to be understood that pharmaceutically acceptable salts, esters, amides, prodrugs, active metabolites, isomers, analogs, etc. of the agent are intended as well as the agent per se.

By “an effective amount” or a “therapeutically effective” amount of an active agent is meant a nontoxic but sufficient amount of the agent to provide a beneficial effect. The amount of active agent that is “effective” will vary from subject to subject, depending on the age and general condition of the individual, the particular active agent or agents, and the like. Unless otherwise indicated, the term “therapeutically effective” amount as used herein is intended to encompass an amount effective for the prevention of an adverse condition and/or the amelioration of an adverse condition, i.e., in addition to an amount effective for the treatment of an adverse condition.

The term “controlled release” refers to an agent-containing formulation or fraction thereof in which release of the agent is not immediate, i.e., with a “controlled release” formulation, administration does not result in immediate release of the agent into an absorption pool. The term is used interchangeably with “nonimmediate release” as defined in Remington: The Science and Practice of Pharmacy, Twenty Third Ed. (Adeboye Adejare (ed.); Amsterdam, The Netherlands; Elsevier Science, 2021). In general, the term “controlled release,” as used herein, refers to “sustained release” rather than to “delayed release” formulations. The term “sustained release” (synonymous with “extended-release”) is used in its conventional sense to refer to a formulation that provides for the gradual release of an agent over a prolonged period of time.

As will be apparent to those of skill in the art upon reading this invention, each of the individual embodiments described and illustrated herein has discrete components and features that may be readily separated from or combined with the features of any of the other several embodiments without departing from the scope or spirit of the present invention. Any recited method can be carried out in the order of events recited or in any other order that is logically possible.

Unless otherwise indicated, the invention is not limited to specific formulation components, modes of administration, chelating agents, manufacturing processes, or the like, as such may vary.

Unless otherwise defined, all technical and scientific terms used herein have the same meaning as commonly understood by one of ordinary skill in the art to which this invention pertains. In the case of conflict, the present document, including definitions, will control.

### Ophthalmic Disorders and Adverse Conditions

The aging process affects all parts of the eye, including the cornea, sclera, trabeculum, iris, lens, vitreous humor, and retina.

The cornea is the eye's outermost layer. It is the clear, dome-shaped surface that covers the front of the eye. The cornea is composed of five layers. The epithelium is a layer of cells that forms the surface. It is only about 5-6 cell layers thick and quickly regenerates when the cornea is injured. If an injury penetrates more deeply into the cornea, scarring may occur and leave opaque areas, causing the cornea to lose its clarity and luster. Immediately below the epithelium is Bowman's membrane, a protective layer that is very tough and difficult to penetrate. The stroma, the thickest layer of the cornea, lies just beneath Bowman's membrane and is composed of tiny collagen fibrils aligned in parallel. This arrangement provides the cornea with its clarity. Descemet's membrane underlies the stroma and is just above the innermost corneal layer, the endothelium. The endothelium is just one cell layer in thickness and serves to pump water from the cornea to the aqueous, keeping it clear. If damaged or diseased, these cells will not regenerate.

As the eye ages, the cornea can become more opaque. Opacification can take many forms. The most common form of opacification affects the periphery of the cornea and is termed “arcus senilis” or “arcus.” This type of opacification initially involves the deposition of lipids into Descemet's membrane. Subsequently, lipids deposit into Bowman's membrane and possibly into the stroma as well. Arcus senilis is usually not visually significant but is a cosmetically noticeable sign of aging. There are other age-related corneal opacifications, however, which may have some visual consequences. These include the central cloudy dystrophy of Francois, which affects the middle layers of the stroma, and posterior crocodile shagreen, which is the central opacification of the posterior stroma. Opacification, by scattering light, results in the progressive reduction of visual contrast and visual acuity.

Opacification of the cornea develops as a result of a number of factors, including, by way of example, degeneration of corneal structure; cross-linking of collagen and other proteins by metalloproteinases; ultraviolet (UV) light damage; oxidation damage, and buildup of substances like calcium salts, protein waste, and excess lipids.

There is no established treatment for slowing or reversing corneal changes other than surgical intervention. For example, opaque structures can be scraped away with a blunt instrument after first removing the epithelium, followed by smoothing and sculpting the corneal surface with a laser beam. In severe cases of corneal scarring and opacification, corneal transplantation has been the only effective approach.

The trabeculum, also referred to as the trabecular meshwork, is a mesh-like structure located at the iris-sclera junction in the anterior chamber of the eye. The trabeculum filters aqueous fluid and controls its flow from the anterior chamber into the canal of Schlemm. As the eye ages, debris and protein-lipid waste may build up and clog the trabecula, a problem that results in increased pressure within the eye, which in turn can lead to glaucoma and damage to the retina, optic nerve, and other structures of the eye. Glaucoma drugs can help reduce this pressure, and surgery can create an artificial opening to bypass the trabeculum and reestablish the flow of liquid out of the vitreous and aqueous humor. There is, however, no known method for preventing a build-up of debris and protein-lipid waste within the trabeculum.

The Iris and Pupil: With age, dilation and constriction of the iris in response to changes in illumination become slower, and its range of motion decreases. Also, the pupil becomes progressively smaller with age, severely restricting the amount of light entering the eye, especially under low-light conditions. The narrowing pupil and the stiffening, slower adaptation, and constriction of the iris over time are largely responsible for the difficulty the aged have in seeing at night and adapting to changes in illumination. The changes in iris shape, stiffness, and adaptability are generally thought to come from fibrosis and cross-linking between structural proteins. Deposits of protein and lipid wastes on the iris over time may also lighten its coloration. Both the light-colored deposits on the iris and the narrowing of the pupil are very noticeable cosmetic markers of age that may have social implications for individuals. There is no standard treatment for any of these changes or for changes in iris coloration with age.

### Cataracts

The opacity of the lens results in an abnormal condition generally known as a cataract. Cataract is a progressive ocular disease, which subsequently leads to lower vision. Most of this ocular disease is age-related senile cataract. The incidence of cataract formation is thought to be 60-70% in persons in their sixties and nearly 100% in persons eighty years or older. However, at the present time, there is no agent that has been clearly proven to inhibit the development of cataracts. Therefore, the development of an effective therapeutic agent has been desired. Presently, the treatment of cataracts depends upon the correction of vision using eyeglasses, contact lenses, or surgical operations such as the insertion of an intra-ocular lens into the capsula lentis after extra-capsular cataract extraction.

In cataract surgery, the incidence of secondary cataracts after surgery has been a problem. Secondary cataract is equated with opacity present on the surface of the remaining posterior capsule following extracapsular cataract extraction. The mechanism of secondary cataracts is mainly as follows. After excising lens epithelial cells (anterior capsule), secondary cataracts result from migration and proliferation of residual lens epithelial cells, which are not completely removed at the time of extraction of the lens cortex, onto the posterior capsule, leading to posterior capsule opacification. In cataract surgery, it is impossible to remove lens epithelial cells completely, and consequently, it is difficult to always prevent secondary cataracts. It is said that the incidence of the above posterior capsule opacification is 40-50% in eyes that do not receive an intracapsular posterior chamber lens implant and 7-20% in eyes that do receive an intracapsular lens implant. Additionally, eye infections categorized as endophthalmitis have also been observed after cataract surgeries.

Disturbances in calcium homeostasis are associated with various forms of cataracts. Ca²⁺-mediated disintegrative globulization of the fiber cells may be responsible for the formation of light-scattering centers during cataractogenesis. Age-related cataract is a multifactorial degenerative disorder in which excess free iron catalyzes the generation of reactive oxygen species (ROS), and elevated intracellular calcium initiates the inflammatory cascade and binds damaged proteins as light-scattering particles in the lens. (Wang L, Bhatnagar A, Ansari N H, Dhir P, Srivastava S K. Mechanism of calcium-induced disintegrative globulization of rat lens fiber cells. Invest Ophthalmol Vis Sci. 1996; 37(5):915-922). This mechanism has been closely associated with the physiological aging process. Cataract is characterized by the loss of lens transparency (caused by the increase in size and number of light-scattering particles) and accompanied by loss of visual function. (Gilliland K O, Freel C D, Lane C W, Fowler W C, Costello M J. Multilamellar bodies as potential scattering particles in human age-related nuclear cataracts. Mol Vis. 2001; 7:120-130. Published 2001 Jun. 22; Gilliland K O, Freel C D, Johnsen S, Craig Fowler W, Costello M J. Distribution, spherical structure and predicted Mie scattering of multilamellar bodies in human age-related nuclear cataracts. Exp Eye Res. 2004; 79(4):563-576. doi:10.1016/j.exer.2004.05.017).

A number of changes can occur in the retina with age. Atherosclerotic buildup and leakage in the retinal arteries can lead to macular degeneration as well as reduction of peripheral vision. The rods and cones can become less sensitive over time as they replenish their pigments more slowly. Progressively, all these effects can reduce vision, ultimately leading to partial or complete blindness. Retinal diseases such as age-related macular degeneration have been hard to cure. Current retinal treatments include laser surgery to stop the leaking of blood vessels in the eye.

As alluded to above, current therapeutic attempts to address many ocular disorders and diseases, including aging-related ocular problems, often involve surgical intervention. Surgical procedures are, of course, invasive, and, furthermore, often do not achieve the desired therapeutic goal. Additionally, surgery can be very expensive and may result in significant undesired after-effects. For example, secondary cataracts may develop after cataract surgery and infections may set in. Endophthalmitis has also been observed after cataract surgery. In addition, advanced surgical techniques are not universally available because they require a very well-developed medical infrastructure. Therefore, it would be of significant advantage to provide straightforward and effective pharmacological therapies that obviate the need for surgery.

Many adverse ocular conditions are associated with the formation, presence, and/or growth of macromolecular aggregates in the eye. Indeed, many pathologies result from or are associated with the deposition and/or aggregation of proteins, other peptidyl species, lipoproteins, lipids, polynucleotides, and other macromolecules throughout the body. For example, Advanced Glycation Endproducts (AGEs) are formed by the binding of glucose or other reducing sugars to proteins, lipoproteins and DNA by a process known as non-enzymatic glycation, followed by cross-linking. These cross-linked macromolecules stiffen connective tissue and lead to tissue damage in the kidney, retina, vascular wall and nerves. AGEs have, in fact, been implicated in the pathogenesis of a variety of debilitating diseases, such as diabetes, atherosclerosis, Alzheimer's, and rheumatoid arthritis, as well as in the normal aging process. Peptidyl deposits are also associated with Alzheimer's disease, sickle cell anemia, multiple myeloma, and prion diseases. Lipids, particularly sterols and sterol esters, represent an additional class of biomolecules that form pathogenic deposits in vivo, including atherosclerotic plaque, gallstones, and the like. To date, no single formulation has been identified as capable of treating a plurality of such disorders.

### Contrast Sensitivity (CS) Decline as a Marker of Progress of Early Cataracts.

Cataract is characterized by the loss of lens transparency (caused by the increase in size and number of light-scattering particles) and accompanied by loss of visual function. (Gilliland K O, et al., Multilamellar bodies as potential scattering particles in human age-related nuclear cataracts. Mol Vis. 2001; 7:120-130. Published 2001 Jun. 22; Gilliland K O, et al., Distribution, spherical structure and predicted Mie scattering of multilamellar bodies in human age-related nuclear cataracts. Exp Eye Res. 2004; 79(4):563-576. doi:10.1016/j.exer.2004.05.017). During the early stages, it has been reported that patients with early cataracts often suffer from a decline in contrast sensitivity (CS) while maintaining relatively good visual acuity (VA). (Fujikado T, et al. Light scattering and optical aberrations as objective parameters to predict visual deterioration in eyes with cataracts. J Cataract Refract Surg. 2004; 30(6):1198-1208. doi:10.1016/j.jcrs.2003.12.023).

CS is a more appropriate visual function measurement than traditional VA measurement for patients with early cataracts, as CS provides important additional information about visual function for performing everyday vision-related tasks that traditional VA measurement does not address. (Jindra-1989) In particular, mesopic CS is most relevant in real-life situations such as driving at night or in foggy/rainy conditions, as well as reading signs, recognizing faces, and reading documents in early twilight or dimly lighted conditions. Mesopic vision is an intermediate stage between photopic and scotopic vision, where both rods and cones are functioning. Contrast is the ratio of the light (visual stimulus) intensity between light and dark areas. The least amount of contrast at which a visual stimulus can be detected is the contrast threshold. CS is the reciprocal of the contrast threshold and varies with the spatial frequency (denoted in cycles per degree of angle) of the patterned visual stimulus. The curve of contrast sensitivity across a range of spatial frequencies is called the contrast sensitivity function (CSF) and is often presented as a graph on a log scale. Area under the log CSF (AULCSF) is a summary metric for total CS across all measured spatial frequencies. Therefore, AULCSF quantifies the entire range of contrast visibility. (Applegate R A, Howland H C, Sharp R P, Cottingham A J, Yee R W. Corneal aberrations and visual performance after radial keratotomy. J Refract Surg. 1998; 14(4):397-407. doi:10.3928/1081-597X-19980701-05).

A well-documented limitation of the Functional Acuity Contrast Test (FACT) makes accurate CS measurements difficult around maximum (ceiling) and minimum (floor) measurable values. (Pesudovs K, et al. Br J Ophthalmol. 2004; 88(1):11-16. doi:10.1136/bjo.88.1.11; Chen Z, et al. Transl Vis Sci Technol. 2021; 10(7):7. doi:10.1167/tvst.10.7.7). Therefore, those eyes with potentially large ceiling and/or floor effects at baseline in any spatial frequency were excluded during analysis. These additional exclusion criteria allowed for more accurate CS measurements with measurable room for improvement and would have the study (subgroup) population be more representative of early cataract patients with loss of CS.

A secondary efficacy endpoint can form the basis for additional indications beyond the primary efficacy endpoint in this study: the mean reduction from baseline in Average Lens Density as measured by densitometric analysis using Scheimpflug and/or OCT imaging after a sufficient time, say, Day 120. A hierarchical analysis is used to test this secondary efficacy endpoint. Scheimpflug tomography uses the Scheimpflug imaging principle, whereby the object and image planes are placed at such an angle to allow the entire object to be focused in a single image. Curvature maps of the cornea are then reconstructed from multiple tomographic images.

### Chelating Agent/Chelator

Chelation is a chemical combination with a metal in complexes in which the metal is part of a ring. An organic ligand is called a chelator or chelating agent, the chelate is a metal complex. The larger the number of ring closures to a metal atom, the more stable the compound. The stability of a chelate is also related to the number of atoms in the chelate ring. Monodentate ligands that have one coordinating atom, like H2O or NH3, are easily broken apart by other chemical processes, whereas polydentate chelators, donating multiple bonds to metal ions, provide more stable complexes. Chlorophyll, a green plant pigment, is a chelate that consists of a central magnesium atom joined with four complex chelating agents (pyrrole ring). Heme is an iron chelate that contains iron (II) ion in the center of the porphyrin. Chelating agents offer a wide range of sequestrants to control metal ions in aqueous systems. By forming stable water-soluble complexes with multivalent metal ions, chelating agents prevent undesired interaction by blocking the normal reactivity of metal ions and/or sequestering and/or transporting them away (e.g., as EDTA does in the body, the iron ion remains reactive while being sequestered and/or transported). EDTA (ethylenediamine tetraacetate) is a good example of a common chelating agent that has nitrogen atoms and short-chain carboxylic groups.

Examples of chelators of iron and calcium include but are not limited to, Diethylene triamine pentaacetic acid (DTPA), ethylene diamine tetraacetic acid (EDTA), nitrilotriacetic acid (NTA), 1,3-propylene diamine tetraacetic acid (PDTA), Ethylene diamine disuccinic acid (EDDS), and ethylene glycol tetraacetic acid (EGTA). Any suitable chelating agent known in the art, which is biologically safe and able to chelate iron, calcium, or other metals, is suitable for the invention.

Compounds useful as chelating agents herein include any compounds that coordinate to or form complexes with a divalent or polyvalent metal cation, thus serving as a sequestrant of such cations. Accordingly, the term “chelating agent” herein includes not only divalent and polyvalent ligands (which are typically referred to as “chelators”) but also monovalent ligands capable of coordinating to or forming complexes with the metal cation.

Suitable biocompatible chelating agents useful in conjunction with the present invention include, without limitation, monomeric polyacids such as EDTA, cyclohexanediamine tetraacetic acid (CDTA), hydroxyethyl ethylenediamine triacetic acid (HEDTA), diethylenetriamine pentaacetic acid (DTPA), dimercaptopropane sulfonic acid (DMPS), dimercaptosuccinic acid (DMSA), amino trimethylene phosphonic acid (ATPA), citric acid, pharmaceutically acceptable salts thereof, and combinations of any of the foregoing. Other exemplary chelating agents include phosphates, e.g., pyrophosphates, tripolyphosphates, and hexametaphosphates.

EDTA is a synthetic amino acid that was first used therapeutically in the 1940s for the treatment of heavy metal poisoning. It is included in the FDA's Inactive Ingredient Approved Drug Products Database for use in various forms including ophthalmic solutions up to 0.13% w/v and up to 0.3% w/v in ophthalmic emulsions. EDTA reduces the concentrations of metal ions, like iron and calcium, present in the eye via chelation.

EDTA and ophthalmologically acceptable EDTA salts are particularly preferred, wherein representative ophthalmologically acceptable EDTA salts are typically selected from diammonium EDTA, disodium EDTA, dipotassium EDTA, triammonium EDTA, trisodium EDTA, tripotassium EDTA, and calcium disodium EDTA.

An exemplary chelator incorporated in compositions of the present invention is Disodium ethylenediaminetetraacetic acid dihydrate (Na₂EDTA·2H₂O); Edetate Disodium Dihydrate; C₁₀H₁₆N₂O₈Na₂·2H₂O.

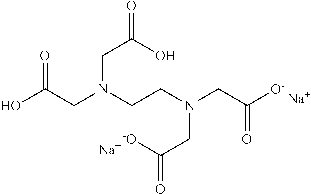

EDTA has been widely used as an agent for chelating metals in biological tissue and blood and has been suggested for inclusion in various formulations. For example, U.S. Pat. No. 6,348,508 to Denick Jr. et al. describes EDTA as a sequestering agent to bind metal ions. In addition to its use as a chelating agent, EDTA has also been widely used as a preservative in place of benzalkonium chloride, as described, for example, in U.S. Pat. No. 6,211,238 to Castillo et al. U.S. Pat. No. 6,265,444 to Bowman et al. discloses the use of EDTA as a preservative and stabilizer. However, EDTA has generally not been applied topically in any significant concentration formulations because of its poor penetration across biological membranes and biofilms, including skin, cell membranes, and even biofilms like dental plaque.

Among the chelating/sequestering materials which may be included in the compositions there may be mentioned biocompatible chelating agents include, without limitation, monomeric polyacids such as EDTA, cyclohexanediamine tetraacetic acid (CDTA), hydroxyethyl ethylenediamine triacetic acid (HEDTA), diethylenetriamine pentaacetic acid (DTPA), dimercaptopropane sulfonic acid (DMPS), dimercaptosuccinic acid (DMSA), amino trimethylene phosphonic acid (ATPA), citric acid, pharmaceutically acceptable salts thereof, and combinations of any of the foregoing.

Other exemplary chelating agents include phosphates, e.g., pyrophosphates, tripolyphosphates, and hexametaphosphates. Other exemplary chelating agents include: phosphates, e.g., pyrophosphates, tripolyphosphates, and hexametaphosphates; chelating antibiotics such as chloroquine and tetracycline; nitrogen-containing chelating agents containing two or more chelating nitrogen atoms within an imino group or in an aromatic ring (e.g., diimines, 2,2′-bipyridines, etc.); and polyamines such as cyclam (1,4,7,11-tetraazacyclotetradecane), N—(C₁-C₃₀ alkyl)-substituted cyclams (e.g., hexadecyclam, tetramethylhexadecylcyclam), diethylenetriamine (DETA), spermine, diethylnorspermine (DENSPM), diethylhomo-spermine (DEHOP), deferoxamine (N′-{5-[Acetyl(hydroxy)amino]pentyl}-N-[5-({4-[(5-aminopentyl)(hydroxy)amino]-4-oxobutanoyl}amino)pentyl]-N-hydroxysuccinamide, or N′-[5-(Acetyl-hydroxy-amino)pentyl]-N-[5-[3-(5-aminopentyl-hydroxy-carbamoyl) propanoylamino]pentyl]-N-hydroxy-butane diamide); also known as desferrioxamine B, desferoxamine B, DFO-B, DFOA, DFB or desferal), deferiprone, pyridoxal isonicotinoyl hydrazone (PIH), salicylaldehyde isonicotinoyl hydrazone (SIH), ethane-1,2-bis(N-1-amino-3-ethylbutyl-3-thiol).

Additional, suitable biocompatible chelating agents which may be useful for the practice of the current disclosure include EDTA-4-aminoquinoline conjugates such as ([2-(Bis-ethoxycarbonylmethyl-amino)-ethyl]-{[2-(7-chloro-quinolin-4-ylamino)-ethylcarbamoyl]-methyl}-amino)-acetic acid ethyl ester, ([2-(Bis-ethoxycarbonylmethyl-amino)-propyl]-{[2-(7-chloro-quinolin-4-ylamino)-ethylcarbamoyl]-methyl}-amino)-acetic acid ethyl ester, ([3-(Bis-ethoxycarbonylmethyl-amino)-propyl]-{[2-(7-chloro-quinolin-4-ylamino)-ethylcarbamoyl]-methyl}-amino)-acetic acid ethyl ester, ([4-(Bis-ethoxycarbonylmethyl-amino)-butyl]-{[2-(7-chloro-quinolin-4-ylamino)-ethylcarbamoyl]-methyl}-amino)-acetic acid ethyl ester, ([2-(Bis-ethoxymethyl-amino)-ethyl]-{[2-(7-chloro-quinolin-4-ylamino)-ethylcarbamoyl]-methyl}-amino)-acetic acid ethyl ester, ([2-(Bis-ethoxymethyl-amino)-propyl]-{[2-(7-chloro-quinolin-4-ylamino)-ethylcarbamoyl]-methyl}-amino)-acetic acid ethyl ester, ([3-(Bis-ethoxymethyl-amino)-propyl]-{[2-(7-chloro-quinolin-4-ylamino)-ethylcarbamoyl]-methyl}-amino)-acetic acid ethyl ester, ([4-(Bis-ethoxymethyl-amino)-butyl]-{[2-(7-chloro-quinolin-4-ylamino)-ethylcarbamoyl]-methyl}-amino)-acetic acid ethyl ester as described in Solomon et al., Med. Chem. 2:133-138, 2006.

Additionally, natural chelators include but are not limited to, citric acid, phytic acid, lactic acid, acetic acid, and their salts. Other natural chelators like but not limited to curcumin (turmeric).

In some embodiments, the chelating agent incorporated in the formulation is a prochelator. A prochelator is any molecule that is converted to a chelator when exposed to the appropriate chemical or physical conditions. For example, BSIH (isonicotinic acid [2-(4,4,5,5-tetramethyl-[1,3,2]dioxaborolan-2-yl)-benzylidene]-hydrazide) prochelators are converted by hydrogen peroxide into SIH (salicylaldehyde isonicotinoyl hydrazone) iron-chelating agents that inhibit iron-catalyzed hydroxyl radical generation.

The inactivated metal ion sequestering agent is sometimes referred to herein as a “prochelator,” although sequestration of metal ions can involve sequestration and complexation processes beyond the scope of chelation per se. The term “prochelator” is analogous to the term “prodrug” insofar as a prodrug is a therapeutically inactive agent until activated in vivo, and the prochelator, as well, is incapable of sequestering metal ions until activated in vivo.

### Transport Enhancer

The instant invention provides eye drops to deliver EDTA into the eye with enhanced penetration. To increase the transport of EDTA into the eye, the surfactant methylsulfonylmethane (MSM) is used as a permeation enhancer.

MSM is a naturally occurring organosulfur zwitterionic surfactant. It is used in Livionex Eye Drops to change surface tension characteristics and allows the permeation of various molecules across charged membranes like the corneal epithelium. EDTA, the active ingredient in Livionex Eye Drops, does not cross negatively charged surfaces like the corneal epithelium. However, when MSM is added to EDTA, the EDTA can cross negatively charged membranes in a concentration-dependent manner. (Zhang-2009). In the instant eye drop formulation, the surfactant MSM enables the penetration of EDTA into the negatively charged epithelium. This allows EDTA to perform its chelation function.

The transport enhancer is selected to facilitate the transport of a chelating agent through the tissues, extra-cellular matrices, and/or cell membranes of a body. An “effective amount” of the transport enhancer represents an amount and concentration within a formulation of the invention that is sufficient to provide a measurable increase in the penetration of a chelating agent through one or more of the sites in a subject than would otherwise be the case without the inclusion of the transport enhancer within the formulation.

In certain instances, the transport enhancer may be present in a formulation of the invention in an amount that ranges from about 0.01 wt. % or less to about 30 wt. % or more, typically in the range of about 0.1 wt. % to about 20 wt. %, more typically in the range of about 1 wt. % to about 11 wt. %, and most typically in the range of about 2 wt. % to about 8 wt. %, for instance, 5 wt. %.

One transport enhancer is generally of the formula (I)

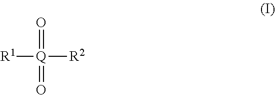

wherein R¹ and R² are independently selected from C₂-C₆ alkyl, C₁-C₆ heteroalkyl, C₆-C₁₄ aralkyl, and C₂-C₁₂ heteroaralkyl, any of which may be substituted, and Q is S or P. Compounds wherein Q is S and R¹ and R² are C₁-C₃ alkyl are preferred, with methylsulfonylmethane (MSM) being the optimal transport enhancer.

Suitable permeation enhancers include methylsulfonylmethane (MSM; also referred to as methyl sulfone) and/or combinations of MSM with dimethylsulfoxide (DMSO). MSM is an odorless, highly water-soluble (34% w/v at 79° F.) white crystalline compound with a melting point of 108-110° C. and a molecular weight of 94.1 g/mol. MSM is thought to serve as a multifunctional agent herein, insofar as the agent not only increases the permeability of biological membranes such as cell membranes, but may also facilitate the transport of one or more formulation components throughout the layers of the skin (i.e., epidermis, dermis and subcutaneous fat layers), as well as across mucus membranes, endothelial layers, and the like. Furthermore, it has been suggested that MSM per se has medicative effects, and can serve as an anti-inflammatory agent as well as an analgesic. It has also been suggested that MSM also acts to improve oxidative metabolism in biological tissues and is a source of organic sulfur, which may assist in the reduction of scarring. MSM additionally possesses beneficial solubilization properties, in that it is soluble in water, as noted above, but exhibits both hydrophilic and hydrophobic properties because of the presence of polar S═O groups and nonpolar methyl groups. The molecular structure of MSM also allows for hydrogen bonding with other molecules, i.e., between the oxygen atom of each S═O group and hydrogen atoms of other molecules, and for the formation of van der Waals associations, i.e., between the methyl groups and nonpolar (e.g., hydrocarbyl) segments of other molecules. The methods and formulations herein may involve the use of two or more permeation enhancers combined.

The phrase “having the formula” or “having the structure” is not intended to be limiting and is used in the same way that the term “comprising” is commonly used. With respect to the above structure, the term “alkyl” refers to a linear, branched, or cyclic saturated hydrocarbon group containing 1 to 6 carbon atoms, such as methyl, ethyl, n-propyl, isopropyl, n-butyl, isobutyl, t-butyl, cyclopentyl, cyclohexyl and the like. If not otherwise indicated, the term “alkyl” includes unsubstituted and substituted alkyl, wherein the substituents may be, for example, halo, hydroxyl, sulfhydryl, alkoxy, acyl, etc. The term “alkoxy” intends an alkyl group bound through a single, terminal ether linkage; that is, an “alkoxy” group may be represented as —O-alkyl where alkyl is as defined above. The term “aryl” refers to an aromatic substituent containing a single aromatic ring or multiple aromatic rings that are fused together, directly linked, or indirectly linked (such that the different aromatic rings are bound to a common group such as a methylene or ethylene moiety). Preferred aryl groups contain 5 to 14 carbon atoms. Exemplary aryl groups contain one aromatic ring or two fused or linked aromatic rings, e.g., phenyl, naphthyl, biphenyl, diphenyl ether, diphenylamine, benzophenone, and the like. “Aryl” includes unsubstituted and substituted aryl, wherein the substituents may be as set forth above with respect to optionally substituted “alkyl” groups. The term “aralkyl” refers to an alkyl group with an aryl substituent, wherein “aryl” and “alkyl” are defined above. Preferred aralkyl groups contain 6 to 14 carbon atoms, and particularly preferred aralkyl groups contain 6 to 8 carbon atoms. Examples of aralkyl groups include, without limitation, benzyl, 2-phenyl-ethyl, 3-phenyl-propyl, 4-phenyl-butyl, 5-phenyl-pentyl, 4-phenylcyclohexyl, 4-benzylcyclohexyl, 4-phenylcyclohexylmethyl, 4-benzylcyclohexylmethyl, and the like. The term “acyl” refers to substituents having the formula —(CO)-alkyl, —(CO)-aryl, or —(CO)-aralkyl, wherein “alkyl,” “aryl, and “aralkyl” are as defined above. The terms “heteroalkyl” and “heteroaralkyl” are used to refer to heteroatom-containing alkyl and aralkyl groups, respectively, i.e., alkyl and aralkyl groups in which one or more carbon atoms is replaced with an atom other than carbon, e.g., nitrogen, oxygen, sulfur, phosphorus or silicon, typically nitrogen, oxygen or sulfur.

In one embodiment, a method is provided for eliminating or reducing the size of an aggregate of macromolecules in the eye. The method involves administering to the eye(s) of a patient a therapeutically effective amount of a sterile ophthalmic formulation comprised of (a) a noncytotoxic chelating agent containing at least three negatively charged chelating atoms, (b) a charge-masking agent containing at least one polar group and (c) a thickening agent HEC at a concentration greater than 0.25%, and inert ophthalmologically acceptable carriers. The polar group of the charge-masking agent contains at least one and preferably at least two heteroatoms having a Pauling electronegativity greater than about 3.00, wherein the heteroatoms are preferably oxygen atoms. The molar ratio of the charge-masking agent to the chelating agent is sufficient to ensure that substantially all negatively charged chelating atoms are associated with at least one of the aforementioned heteroatoms on the charge-masking agent. The formulation may be applied to the eye in any form suitable for ocular drug administration, e.g., as a solution or suspension for administration as eye drops or eye washes, as an ointment, or in an ocular insert that can be implanted in the conjunctiva, sclera, pars plana, anterior segment, or posterior segment of the eye. Such inserts provide controlled release of the formulation to the ocular surface, typically sustained release over an extended period of time.

The formulation may also be applied to the skin around the eye for penetration therethrough, insofar as the compound used as the charge-masking agent, e.g., methylsulfonylmethane, also serves as a penetration enhancer allowing permeation of the formulation through the skin.

In another embodiment, the formulation is effective in alleviating dry eye symptoms, especially dry eyes associated with inflammation, and can be used to treat dry eyes. Subjects with extremely dry eyes, such as those with Sjogren's Syndrome, may yet experience some stinging sensation due to the extreme dryness of their eyes.

Accordingly, the chelating agent is multifunctional in the context of the present invention insofar as the agent serves to decrease unwanted proteins or peptides, prevent the formation of mineral deposits, and/or reduce mineral deposits that have already formed, and reduce calcification in addition to acting as a preservative and stabilizing agent.

The formulation also includes an effective amount of a transport enhancer that facilitates penetration of the formulation components through cell membranes, tissues, and extracellular matrices. The “effective amount” of the transport enhancer represents a concentration that is sufficient to provide a measurable increase in penetration of one or more of the formulation components through membranes, tissues, and extracellular matrices as just described. Suitable transport enhancers include, by way of example, methylsulfonylmethane (MSM; also referred to as methyl sulfone), combinations of MSM with dimethylsulfoxide (DMSO), or a combination of MSM and, in a less preferred embodiment, DMSO, with MSM particularly preferred.

MSM is an odorless, highly water-soluble (34% w/v @ 79° F.) white crystalline compound with a melting point of 108-110° C. and a 94.1 g/mol molecular weight. MSM serves as a multifunctional agent herein insofar as the agent not only increases cell membrane permeability but also acts as a “transport facilitating agent” (TFA) that aids in the transport of one or more formulation components to the eye. Further, MSM per se provides medicative effects and can serve as an anti-inflammatory agent as well as an analgesic. MSM also acts to improve oxidative metabolism in biological tissues and is a source of organic sulfur, which assists in the reduction of scarring. MSM additionally possesses unique and beneficial solubilization properties, in that it is soluble in water, as noted above, but exhibits both hydrophilic and hydrophobic properties because of the presence of polar S═O groups and nonpolar methyl groups. The molecular structure of MSM also allows for hydrogen bonding with other molecules, i.e., between the oxygen atom of each S═O group and hydrogen atoms of other molecules, and for the formation of van der Waal associations, i.e., between the methyl groups and nonpolar (e.g., hydrocarbyl) segments of other molecules. Ideally, the concentration of MSM in the present formulations is in the range of about 0.1 wt. % to 40 wt. %, or from about 1 wt. % to about 4, 5, 6, 7, 8, 10, 15 wt. %, and preferably between about 1.5 wt. % to 8.0 wt. %.

Other optional additives in the present formulations include secondary enhancers, i.e., one or more additional transport enhancers. For example, the formulation of the invention can contain added DMSO. Since MSM is a metabolite of DMSO (i.e., DMSO is enzymatically converted to MSM), incorporating DMSO into an MSM-containing formulation of the invention will tend to gradually increase the fraction of MSM in the formulation. DMSO also serves as a free radical scavenger, thereby reducing the potential for oxidative damage. If DMSO is added as a secondary enhancer, the amount is preferably in the range of about 1.0 wt. % to 2.0 wt. % of the formulation and the weight ratio of MSM to DMSO is typically in the range of about 1:50 to about 50:1.

The formulation may further comprise a second transport enhancer that enhances the permeability of the chelator. Compounds of this class include but are not limited to a glycol ether, alkyl ethers of diethylene glycol, butylene glycol, dipropylene glycol, ethylene glycol, or propylene glycol. Biologically active compounds conjugated with polyoxyalkylenes can provide enhanced biocompatibility for the compound. (U.S. Pat. Nos. 5,366,735 and 6,280,745). Polyethylene glycol is one of the best biocompatible polymers to conjugate with a biologically active compound such as a drug to produce a conjugate that has improved properties such as compatible solubility characteristics and improved surface compatibility. Polyethylene glycol (PEG) is a linear polyoxyalkylene compound terminated at the ends thereof with hydroxyl groups and generally represented by the formula HO(CH₂CH₂O)ₙH. In some embodiments, the formulation comprises a substituted polyethylene glycol compound or polyethylene glycol.

One exemplary permeation enhancer is propylene glycol (PG), which is a synthetic liquid substance that absorbs water. The Food and Drug Administration (FDA) has classified propylene glycol as an additive that is “generally recognized as safe”. Propylene glycol (IUPAC name: propane-1,2-diol) is a viscous, colorless liquid that is nearly odorless but possesses a faintly sweet taste. Its chemical formula is CH₃CH(OH)CH₂OH. As it contains two alcohol groups, it is classed as a diol. It is miscible with a broad range of solvents, including water. In general, glycols are non-irritating and have very low volatility. Other names for propylene glycol are 1,2-dihydroxypropane, 1,2-propanediol, methyl glycol, and trimethyl glycol.

In optional embodiments, an excipient or solubilizing agent is added to the formulation to facilitate the solubility of the chelator. Otherwise known as polysorbates, Tweens® are typically used as surfactants for dispersing hydrophobic particles in aqueous solutions. Tweens are a condensate of sorbitol fatty acid ester and ethylene oxide and are either soluble or dispersible in water.

Polysorbate 80 (also known as polyoxyethylene-sorbitan-20 mono-oleate or Tween® 80) is a non-ionic detergent that can be incorporated into the compositions of the current invention. Tween® 80 is an ester of sorbitan and oleic acid. It is a versatile compound commonly employed in various biological applications. Additionally, Tween® 80 finds use in stabilizing proteins and facilitating protein membrane studies. Its effectiveness stems from its membership in the polysorbate group, characterized by combining sorbitol and sorbitan oleic esters with polyethylene glycol chains. These structural features contribute to Tween® 80's emulsifying capabilities. In some embodiments, Tween® 20 (Polysorbate 20, Polyoxyethlenesorbitan Monolaurate 20), a non-ionic surfactant, is used to lower the surface tension of aqueous chelator solutions.

### Formulations

A variety of means can be used to formulate the compositions of the invention. Techniques for formulation and administration may be found in “Remington: The Science and Practice of Pharmacy,” Twenty Third Ed. (Adeboye Adejare (ed.); Amsterdam, The Netherlands; Elsevier Science, 2021). For human or animal administration, preparations should meet sterility, pyrogenicity, general safety, and purity standards comparable to those required by the FDA. Administration of the pharmaceutical formulation can be performed in a variety of ways, as described herein.

Other additives for incorporation into the formulations that are at least partially aqueous include, without limitation, thickeners, isotonic agents, buffering agents, and preservatives, providing that any such excipients do not interact in an adverse manner with any of the formulation's other components. It should also be noted that preservatives are generally unnecessary because the selected chelating agent serves as a preservative. Suitable thickeners will be known to those of ordinary skill in the art of formulation, and include, by way of example, cellulosic polymers such as methylcellulose (MC), hydroxyethylcellulose (HEC), hydroxypropylcellulose (HPC), hydroxypropyl-methylcellulose (HPMC), and sodium carboxymethylcellulose (NaCMC), and other swellable hydrophilic polymers such as polyvinyl alcohol (PVA), hyaluronic acid or a salt thereof (e.g., sodium hyaluronate), and crosslinked acrylic acid polymers commonly referred to as “carbomers” (and available from B.F. Goodrich as Carbopol® polymers) added to a final concentration of to a final concentration of 0.25% to 10.0%, preferably 0.25%-5.0% w/w, and more preferably 0.3%-1.0% w/w.

Guar gum, also called guaran, is a galactomannan polysaccharide extracted from guar beans with thickening and stabilizing properties. In addition to guar gum's effects on viscosity, its high ability to flow or deform gives it favorable rheological properties. Hydroxypropyl Guar Gum, or HPG, is formed by the etherification reaction with non-ionic propylene oxide reagent with guar gum. This modification of guar gum severely improves its properties, like alkaline stability, hydrophobicity, solubility, biostability, etc.

In one embodiment, the eye drops were made with varying concentrations of the thickener HEC and tested in human eyes to estimate their residence times. In eye drops made without HEC, subjects can feel the presence of the EDTA in the nasal passage within 5 seconds of applying the eye drops. The time interval between the application of the eye drops and the perception of the presence of the EDTA in the nasal passage indicates the residence time in the eye. Surprisingly, adding HEC to a final concentration of 0.25% to 1.0% increased residence time to 25 to 125 seconds, respectively. This indicated a 6× to 30× increase in residence time in the presence of the thickener HEC. Further, the addition of thickeners such as HEC increased permeation of an active agent through the membrane of the eye.

Any suitable isotonic agents and buffering agents commonly used in ophthalmic formulations may be used, provided the pH of the formulation is maintained in the range of about 4.5 to about 9.0, preferably in the range of about 6.8 to about 7.8, and optimally at a pH of about 7.4. Preferred buffering agents include carbonates such as sodium and potassium bicarbonate.

However, an effective thickening agent must be used in an amount that also exhibits the key properties of enabling uses of lower concentrations of chelator/MSM combination to achieve significant effect without causing symptoms of discomfort in the eye, such as severe stinging.

The formulations of the invention also include a pharmaceutically acceptable ophthalmic carrier or vehicle, which will depend on the particular type of formulation. For example, the formulations of the invention can be provided as an ophthalmic solution or suspension, in which case the carrier is at least partially aqueous. Ideally, ophthalmic solutions, which may be administered as eye drops, are aqueous solutions. The formulations may also be ointments, in which case the pharmaceutically acceptable carrier is composed of an ointment base. Preferred ointment bases herein have a melting or softening point close to body temperature, and any ointment bases commonly used in ophthalmic preparations may be advantageously employed. Common ointment bases include petrolatum and mixtures of petrolatum and mineral oil. Suitable pharmaceutical formulations and dosage forms may be prepared using conventional methods known to those in the field of pharmaceutical formulation and described in the pertinent texts and literature, e.g., in Remington: The Science and Practice of Pharmacy, cited previously herein.

The formulations of the invention may also be prepared as a hydrogel, dispersion, or colloidal suspension. Hydrogels are formed by the incorporation of a swellable, gel-forming polymer such as those set forth above as suitable thickening agents (i.e., MC, HEC, HPC, HPMC, Na-CMC, PVA, or hyaluronic acid or a salt thereof, e.g., sodium hyaluronate), except that a formulation referred to in the art as a “hydrogel” typically has a higher viscosity than a formulation referred to as a “thickened” solution or suspension. In contrast to such preformed hydrogels, a formulation may also be prepared so as to form a hydrogel in situ following application into the eye. Such gels are liquid at room temperature but gel at higher temperatures (and thus termed “thermoreversible” hydrogels), such as when placed in contact with body fluids. Biocompatible polymers that impart this property include acrylic acid polymers and copolymers, N-isopropylacrylamide derivatives, and ABA block copolymers of ethylene oxide and propylene oxide (conventionally referred to as “poloxamers” and available under the Pluronic® trade name from BASF-Wyandotte). The formulations can also be prepared in the form of a dispersion or colloidal suspension. Preferred dispersions are liposomal, in which case the formulation is enclosed within “liposomes,” microscopic vesicles composed of alternating aqueous compartments and lipid bilayers. Colloidal suspensions are generally formed from microparticles, i.e., from microspheres, nanospheres, microcapsules, or nanocapsules, wherein microspheres and nanospheres are generally monolithic particles of a polymer matrix in which the formulation is trapped, adsorbed, or otherwise contained, while with microcapsules and nanocapsules, the formulation is actually encapsulated. The upper limit for the size of these microparticles is about 5 μm to about 10 μm.

The formulations may also be incorporated into a sterile ocular insert that provides for controlled release of the formulation over an extended time period, generally in the range of about 12 hours to 60 days, and possibly up to 12 months or more, following implantation of the insert into the conjunctiva, sclera, or pars plana, or into the anterior segment or posterior segment of the eye. One type of ocular insert is an implant in the form of a monolithic polymer matrix that gradually releases the formulation to the eye through diffusion and/or matrix degradation. With such an insert, it is preferred that the polymer be completely soluble and or biodegradable (i.e., physically or enzymatically eroded in the eye) so that removal of the insert is unnecessary. These types of inserts are well known in the art and are typically composed of a water-swellable, gel-forming polymer such as collagen, polyvinyl alcohol, or a cellulosic polymer. Another type of insert that can be used to deliver the present formulation is a diffusional implant, which is contained in a central reservoir enclosed within a permeable polymer membrane that allows for gradual diffusion of the formulation out of the implant. Osmotic inserts may also be used, i.e., implants in which the formulation is released as a result of an increase in osmotic pressure within the implant following application to the eye and subsequent absorption of lachrymal fluid.

The chelating agent may be administered, if desired, in the form of a salt, ester, crystalline form, hydrate, or the like, provided it is pharmaceutically acceptable. Salts, esters, etc. may be prepared using standard procedures known to those skilled in the art of synthetic organic chemistry and described, for example, by J. March, Advanced Organic Chemistry: Reactions, Mechanisms and Structure, 4th Ed. (New York: Wiley-Interscience, 1992).

The amount of chelating agent administered will depend on a number of factors and will vary from subject to subject and depend on the particular chelating agent, the particular disorder or condition being treated, the severity of the symptoms, the subject's age, weight, and general condition, and the judgment of the prescribing physician. The term “dosage form” denotes any form of a pharmaceutical composition that contains an amount of chelating agent and transport enhancer sufficient to achieve a therapeutic effect with a single administration or multiple administrations. The frequency of administration that will provide the most effective results in an efficient manner without overdosing will vary with the characteristics of the particular active agent, including both its pharmacological characteristics and its physical characteristics, such as hydrophilicity.

### EXAMPLES

The following examples are put forth so as to provide those skilled in the art with a complete invention and description of how to make and use embodiments in accordance with the invention and are not intended to limit the scope of what the inventors regard as their discovery. Efforts have been made to ensure accuracy with respect to numbers used (e.g., amounts, temperature, etc.), but some experimental errors and deviations should be accounted for. Unless indicated otherwise, parts are parts by weight, molecular weight is weight-average molecular weight, temperature is degrees Celsius, and pressure is at or near atmospheric.

### Example 1: Exemplary Eye Drop Formulations According to the Invention

An exemplary formulation is prepared as follows: High-purity de-ionized (DI) water (500 ml) was filtered via a 0.2 micrometer filter. MSM, EDTA, PEG, and (optionally) Tween 80 were added to filtered deionized water and mixed until visual transparency was achieved, indicating dissolution. The mixture was poured into 10 mL bottles, each with a dropper cap. On a weight percent basis, the eye drops had the following composition: MSM: 5.4% w/w; di-sodium EDTA: 2.6% w/w; PG: 5% w/w; Tween 80: 1% w/w.

In one embodiment, the eye drops had the following composition by weight: EDTA: 2.6%; MSM: 5.4%; Propylene Glycol: 5%; Polysorbate 80:1.5%; Hydroxyethyl Cellulose (HEC): 0.3%: Guar Gum or Hydroxypropyl Guar: 0.1%; deionized water; and the pH adjusted to about 7.4 with sodium hydroxide.

### Example 2: Enhanced Penetration of Chelator in the Presence of PEG

The following table shows the Pharmacokinetics of topically applied 14C-labeled EDTA with MSM, with and without Propylene Glycol (PEG) in rat ocular tissues and brain at 30 min post instillation. The entry into the brain is via the optic nerve and/or the BBB. With the addition of 5% PG, there is an increase of 27% in the eye and f>100% in the brain. (M/E: MSM+EDTA; PG: propylene glycol).

TABLE 1

| | | ¹⁴C counts per minute (cpm) | | | |
|---|---|---|---|---|---|
| | | M/E | | M/E + PG | |
| | | Eye | Brain | Eye | Brain |
| 30 minutes | Mean | 1122 | 230 | 1428 | 485 |
| | Sd | 197 | 53 | 251 | 65 |
| | n | 10 | 10 | 10 | 10 |
| | T stat | | | 3.03 | 9.615 |
| | df | | | 14.5 | 15.2 |
| | P value | | | 0.0084 | <0.0001 |

### Example 3: Efficacy of Topical 2.6% w/w EDTA Ophthalmic Solutions

Purpose: To explore the efficacy of topical 2.6% EDTA ophthalmic solution (C-KAD) as a treatment to improve visual function for the subgroup of patients with loss of contrast sensitivity (CS) due to early-stage age-related cataracts.

Design: Randomized, double-blinded, placebo-controlled, multicenter phase ½ clinical trial.

Methods: Both eyes of subjects in the intent-to-treat population, with mesopic CS scores between 1 and 7 grating patches (range 0-9, each patch representing 0.15 log CS), at baseline in all five frequencies, were included. The proportion of eyes with clinically significant mesopic CS improvement and mean changes in mesopic CS at spatial frequencies between 1.5 to 18 cycles per degree (cpd) and summary metrics of area under the log CS function (AULCSF) were analyzed. Other exploratory outcomes analyzed included best-corrected visual acuity (BCVA) and lens density for a smaller subgroup of eyes for which Scheimpflug images were available.

Results: Forty-one subject eyes were included in the subgroup analysis (C-KAD n=21, placebo n=20). The primary endpoint of the proportion of eyes with mesopic CS improvements ≥0.30 log CS (equivalent to 50% CS improvement) in at least two of the five spatial frequencies was significantly greater for C-KAD (66.7% versus 35.0% for placebo, P=0.043) at Day 120. C-KAD met the primary protocol endpoint in this subgroup analysis. The proportion of eyes achieving ≥0.30 log CS improvement (mesopic) as measured in AULCSF was also significantly greater for C-KAD, with 42.9% compared to 15.0% for placebo (P=0.050) at Day 120.

The mean change in AULCSF (mesopic) was significantly larger for C-KAD, with 0.25 log CS improvement, versus placebo with 0.06 log CS improvement (P=0.020) at Day 120. C-KAD also showed significant mesopic CS improvements at spatial frequencies 3 and 6 cpd, with 0.28 log CS (P=0.004) and 0.31 log CS (P=0.047) versus placebo at Day 120. Positive BCVA trends and statistical significance in lens density were also observed.

Conclusions: A significant treatment effect of C-KAD in visual function and vision quality was observed consistently. These promising results suggest a novel, non-invasive pharmacological treatment to improve vision in patients with early-stage cataracts. All publications and patent applications cited in this specification are herein incorporated by reference as if each individual publication or patent application were specifically and individually indicated to be incorporated by reference.

### Example 4: Mesopic Contrast Sensitivity (CS) Improvements

111 patients (222 subject eyes) with loss of CS due to low-grade cataracts in both eyes were enrolled, from which 41 eyes in 29 patients (21 eyes in 15 patients for C-KAD and 20 eyes in 14 patients for placebo) were included in this analysis dataset.

A change of 0.30 log CS is equivalent to 50% contrast sensitivity improvement and is generally considered as clinically meaningful. (Nixon-2017) The proportion of subject eyes showing ≥0.30 log CS improvement in at least two spatial frequencies was significantly greater for C-KAD, with 66.7% (14/21), than for placebo, with 35.0% ( 7/20), at Day 120 (P=0.043) (Table 2).

TABLE 2
Proportion of Subject Eyes with ≥0.30 LogCS Improvement
(Mesopic) in at least Two Spatial Frequencies

| | Day 30 | | Day 60 | | Day 90 | | Day 120 | |
|---|---|---|---|---|---|---|---|---|
| C-KAD (n = 21) | 33.3% | (7/21) | 38.1% | (8/21) | 52.4% | (11/21) | 66.7% | (14/21) |
| Placebo (n = 20) | 5.0% | (1/20) | 15.0% | (3/20) | 10.0% | (2/20) | 35.0% | (7/20) |

| | | | | |
|---|---|---|---|---|
| Difference | 28.3% | 23.1% | 42.4% | 31.7% |
| P value (chi-square) | .022^{a} | .095 | .004^{a} | .043^{a} |

^{a}Statistically significant with P < .05.

The proportions of subject eyes showing ≥0.30 log CS improvement were greater for C-KAD across all spatial frequencies, with a statistically significant difference at 18 cpd, where the proportion was 42.9% (9/21) for C-KAD compared to 5.0% ( 1/20) for placebo at Day 120 (P=0.005) (Table 3). When comparing AULCSF values, the proportion was also significantly greater for C-KAD, with 42.9% (9/21), than for placebo, with 15.0% ( 3/20) at Day 120 (P=0.050) (Table 3).

TABLE 3
Proportion of Subject Eyes with ≥ 0.30 LogCS
Improvement (Mesopic) at Day 120

| | 1.5 cpd | 3 cpd | 6 cpd | 12 cpd | 18 cpd | AULCSF |
|---|---|---|---|---|---|---|
| C-KAD (n = 21) | 47.6% | 47.6% | 47.6% | 33.3% | 42.9% | 42.9% |
| | (10/21) | (10/21) | (10/21) | (7/21) | (9/21) | (9/21) |
| Placebo (n = 20) | 40.0% | 20.0% | 25.0% | 15.0% | 5.0% | 15.0% |
| | (8/20) | (4/20) | (5/20) | (3/20) | (1/20) | (3/20) |
| Difference | 7.6% | 27.6% | 22.6% | 18.3% | 37.9% | 27.9% |
| P value (chi- | .623 | .062 | .133 | .172 | .005^{a} | .050^{a} |
| square) | | | | | | |

^{a}Statistically significant with P ≤ .05.

In the C-KAD group, mean CS improved significantly from baseline to Day 120 at all spatial frequencies except for 18 cpd. The differences between C-KAD and placebo were significant at 3 cpd (0.28 log CS vs. 0.13 log CS, P=0.004) and at 6 cpd (0.31 log CS vs. 0.09 log CS, P=0.047), with a trend toward statistical significance at other spatial frequencies including at 18 cpd, where CS decline was alleviated in the C-KAD group (Table 4). More importantly, in terms of overall CS, as measured in AULCSF, C-KAD demonstrated a significantly greater improvement of 0.25 log CS versus 0.06 log CS in placebo (P=0.020).

TABLE 4
Mesopic Contrast Sensitivity (Mean ± SD in logCS)

| | 1.5 cpd | 3 cpd | 6 cpd | 12 cpd | 18 cpd | AULCSF |
|---|---|---|---|---|---|---|
| C-KAD (n = 21) | | | | | | |
| Baseline | 1.61 ± 0.11 | 1.69 ± 0.22 | 1.51 ± 0.23 | 1.10 ± 0.16 | 0.69 ± 0.10 | 1.55 ± 0.17 |
| Day 120 | 1.84 ± 0.17 | 1.97 ± 0.16 | 1.82 ± 0.28 | 1.23 ± 0.39 | 0.60 ± 0.47 | 1.79 ± 0.25 |
| Change | 0.23 ± 0.13 | 0.28 ± 0.22 | 0.31 ± 0.34 | 0.13 ± 0.34 | −0.09 ± 0.51 | 0.25 ± 0.24 |
| Placebo (n = 20) | | | | | | |
| Baseline | 1.58 ± 0.13 | 1.68 ± 0.19 | 1.57 ± 0.22 | 1.12 ± 0.17 | 0.72 ± 0.12 | 1.57 ± 0.18 |
| Day 120 | 1.75 ± 0.17 | 1.81 ± 0.19 | 1.66 ± 0.29 | 1.05 ± 0.50 | 0.44 ± 0.43 | 1.62 ± 0.28 |
| Change | 0.17 ± 0.16 | 0.13 ± 0.21 | 0.09 ± 0.29 | −0.07 ± 0.47 | −0.28 ± 0.39 | 0.06 ± 0.26 |
| Mean Difference | 0.06 | 0.15 | 0.22 | 0.20 | 0.19 | 0.19 |
| 95% CI | −0.02, 0.16 | 0.06, 0.26 | 0.00, 0.36 | −0.06, 0.46 | −0.12, 0.46 | 0.03, 0.34 |
| P value | .131 | .004ª | .047ª | .127 | .231 | .020ª |

^{a}Statistically significant with P < .05.

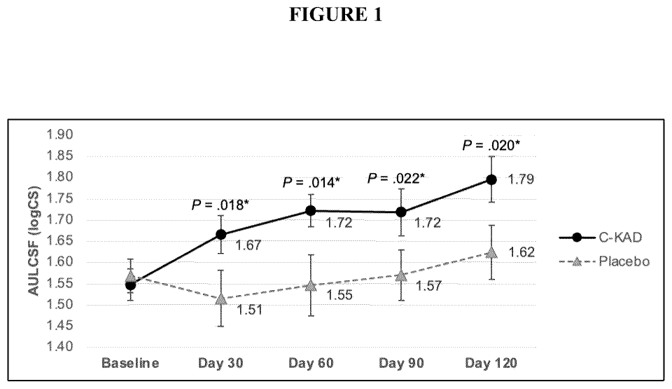

C-KAD consistently outperformed placebo in AULCSF during the treatment duration, and the mean differences were statistically significant in favor of C-KAD from Day 30 through Day 120 (FIG. 1).

FIG. 1 shows the area under the Log Contrast Sensitivity Function (Mean±SE in log CS). Mean area under the log contrast sensitivity function (AULCSF) under mesopic condition as a summary metric for overall contrast sensitivity function (CSF) is presented from baseline to Day 120 for C-KAD and placebo groups. AULCSF allows for quantitative comparison of overall CSFs over time and/or between groups. (ª Statistically significant with P<0.05).

Visual Acuity (VA): Mean BCVA changes from baseline to Day 120 were 5.48 letters for C-KAD and 4.00 letters for placebo. To assess if a lower baseline BCVA might contribute to a greater improvement in BCVA (as they have more room for improvement), further analyses were performed by grouping based on baseline BCVA. The mean BCVA change for those eyes with baseline BCVA of fewer than 85 letters (less than 20/20 vision) was 7.50 letters for C-KAD and 5.50 letters for placebo but did not approach statistical significance. BCVA letter improvements were inversely proportional to the baseline BCVA, with greater letter improvements in eyes with worse baseline visual acuity. Similar findings were reported in previous studies in patients with age-related macular degeneration (AMD), where eyes with better baseline vision were less likely to improve or eyes with worse baseline vision were more likely to improve. (Williams-2011)

Lens Density: Scheimpflug images were captured during CK-0103 for a different subgroup of 17 subject eyes (C-KAD n=9, placebo n=8) and used for densitometric analysis. The mean lens density (ALD) as measured in pixel intensity units of grayscale (ranging from 0 to 255) using ImageJ software and converted to a percentage (ranging from 0% to 100%) decreased from 16.51% at baseline to 16.41% at Day 120 in C-KAD and increased from 16.11% at baseline to 17.09% at Day 120 in placebo (P=0.040) (Table 5).

The topical administration of C-KAD improved visual function for patients with early-stage cataracts by making EDTA more bioavailable to chelate calcium out of the light-scattering particles, thereby reducing the number and size of the light-scattering particles in the lens. The treatment effect of C-KAD was observed by mesopic CS rather than VA since these patients often have a loss of CS (particularly under mesopic conditions) while maintaining relatively good (close to normal) VA (as measured under photopic conditions). Further, CS measurements are much more sensitive and can detect early visual function changes compared to VA measurements. (Owsley C. Contrast sensitivity. Ophthalmol Clin North Am. 2003; 16 (2): 171-177). Given the demonstrated significant improvement in mesopic CS and a positive trend in VA improvement, this analysis provides evidence that C-KAD is a novel and non-invasive treatment for early-stage, age-related cataracts.

### Example 5: Statistical Significance of C-KAD Treatment

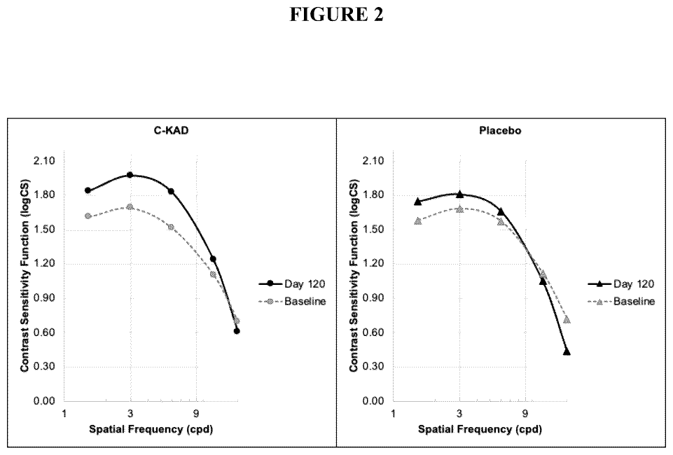

The statistically significant C-KAD treatment effect was demonstrated not only by the responder analysis based on the proportion of subject eyes achieving clinically meaningful (≥0.30 log CS) improvements at ≥2 spatial frequencies in mesopic CS but also by the mean changes of mesopic CS as measured in AULCSF (across all spatial frequencies), as well as at spatial frequencies 3 and 6 cpd, where contrast sensitivity function generally has its peak sensitivity. Peak CS and CS at low to intermediate spatial frequencies are essential in real-life daily activities such as mobility (e.g., walking, driving) and facial recognition. By improving CS at lower to intermediate (1.5, 3, and 6 cpd) spatial frequencies while preventing and/or minimizing contrast sensitivity decline at higher frequencies (12 and 18 cpd), the use of C-KAD resulted in a significantly greater overall improvement in CS function when compared to placebo. (FIG. 2). As a result, at Day 120, the mean mesopic CS at all spatial frequencies for C-KAD fell well within the normative range of older adults without cataracts. (Ginsburg-2006, Stalin-2020). Overall CS improvement as measured in AULCSF for the C-KAD group, 0.25 log CS at Day 120 (FIG. 2), is comparable to a previously described difference of 0.17 log CS between cataract and control groups.

FIG. 2 shows the mean Contrast Sensitivity Function. The contrast sensitivity function (CSF) is plotted as a curve with the mean log CS on the Y-axis and spatial frequencies from 1.5 to 18 cpd on the X-axis on a log scale at baseline and Day 120 for C-KAD and placebo groups. CSF presents contrast sensitivity as a function of spatial frequency.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claim.

What is claimed is:

1. An ophthalmic formulation for non-invasive treatment of age-related cataract, the formulation comprising:
(a) a chelating agent or salts thereof at a concentration of about 0.1% to 15% w/w;
(b) a first permeation enhancer, which is a charge-masking agent methylsulfonylmethane (MSM) at a concentration of about 0.1% to 40% w/w;
(c) a second permeation enhancer, which is a polyoxyalkylene compound at a concentration of about 0.1%-10% w/w;
(d) a thickening agent at a concentration of about 0.25% to 10.0%;
(d) a surfactant for enhancing solubility of the chelating agent at a concentration of about 0.10% to 15.0%; and
(e) pharmaceutically acceptable inactive excipients;
wherein the chelating agent and the permeation enhancers are present in a proportion effective to chelate iron and calcium ions, and
wherein permeation and retention of the chelator in an intraocular space is increased to a level and/or time sufficient to alleviate the symptoms of age-related cataracts.

2. The formulation of claim 1, wherein the proportion of MSM to the chelator is within the range of 10:1 to 1:20 w/w.

3. The formulation of claim 1, wherein the polyoxyalkylene compound is selected from the group consisting of glycol ether, alkyl ethers of diethylene glycol, butylene glycol, dipropylene glycol, ethylene glycol, polyethylene glycol (PEG), propylene glycol (PG), and derivatives thereof.

4. The formulation of claim 1, wherein the thickening agent is a cellulosic polymer selected from the group consisting of methylcellulose (MC), hydroxyethylcellulose (HEC), hydroxypropylcellulose (HPC), hydroxypropyl-methylcellulose (HPMC), sodium carboxymethylcellulose (NaCMC), swellable hydrophilic polymers, polyvinyl alcohol (PVA), hyaluronic acid or a salt thereof and crosslinked acrylic acid polymers.

5. The formulation of claim 1, wherein the surfactant for enhancing solubility of the chelating agent is a polysorbate surfactant selected from TWEEN®80 (2-[2-[3,4-bis(2-hydroxyethoxy)oxolan-2-yl]-2-(2-hydroxyethoxy)ethoxy]ethyl octadec-9-enoate) and TWEEN®20 (2-[2-[3,4-bis(2-hydroxyethoxy)oxolan-2-yl]-2-(2-hydroxyethoxy)ethoxy]ethyl dodecanoate).

6. The formulation of claim 1, wherein the chelating agent is selected from ethylenediamine tetraacetic acid (EDTA), ethylene glycol tetraacetic acid (EGTA), cyclohexanediamine tetraacetic acid (CDTA), hydroxyethyl ethylenediamine triacetic acid (HEDTA), diethylenetriamine pentaacetic acid (DTPA), dimercaptopropane sulfonic acid (DMPS), dimercaptosuccinic acid (DMSA), amino trimethylene phosphonic acid (ATPA), nitrilotriacetic acid (NTA), 1,3-propylene diamine tetraacetic acid (PDTA), ethylene diamine disuccinic acid (EDDS), and ethylene glycol tetraacetic acid (EGTA) and acceptable salts thereof, and any combinations thereof.

7. The formulation of claim 6, wherein the EDTA salt is selected from diammonium EDTA, disodium EDTA, dipotassium EDTA, triammonium EDTA, trisodium EDTA, tripotassium EDTA, tetrasodium EDTA, tetrapotassium EDTA, calcium disodium EDTA, and combinations thereof.

8. The formulation of claim 1, wherein the chelating agent is ethylenediamine tetraacetic acid (EDTA) or salt thereof at a concentration of about 2.6% w/w.

9. The formulation of claim 1, further comprising guar gum or hydroxypropyl guar at 0.1% w/w.

10. The formulation of claim 9, wherein the formulation comprises: MSM at 5.4% w/w; di-sodium EDTA at 2.6% w/w; propylene glycol at 5.0% w/w, hydroxyethylcellulose (HEC) at 0.3%, TWEEN®80 (polysorbate 80) at 15.0% w/w, and optionally, guar gum or hydroxypropyl guar at 0.1% w/w.

11. A method for treating age-related cataracts, comprising administering to the eye of a subject in need thereof a formulation comprising:
(a) a chelating agent or salts thereof at a concentration of about 0.1% to 15% w/w;
(b) a first permeation enhancer, which is a charge-masking agent methylsulfonylmethane (MSM) at a concentration of about 0.1% to 40% w/w;
(c) a second permeation enhancer, which is a polyoxyalkylene compound at a concentration of about 0.1%-10% w/w;
(d) a thickening agent at a concentration of about 0.25% to 10.0%;
(d) a surfactant for enhancing solubility of the chelating agent at a concentration of about 1.0% to 15.0%; and
(e) pharmaceutically acceptable inactive excipients;
wherein the chelating agent and the permeation enhancers are present in a proportion effective to chelate iron and calcium ions.

12. The method of claim 11, wherein the polyoxyalkylene compound is selected from the group consisting of glycol ether, alkyl ethers of diethylene glycol, butylene glycol, dipropylene glycol, ethylene glycol, polyethylene glycol (PEG), propylene glycol (PG) and derivatives thereof.

13. The method of claim 11, wherein the thickening agent is a cellulosic polymer selected from the group consisting of methylcellulose (MC), hydroxyethylcellulose (HEC), hydroxypropylcellulose (HPC), hydroxypropyl-methylcellulose (HPMC), and sodium carboxymethylcellulose (NaCMC), swellable hydrophilic polymers, polyvinyl alcohol (PVA), hyaluronic acid or a salt thereof and crosslinked acrylic acid polymers.

14. The method of claim 11, wherein the surfactant for enhancing solubility of the chelating agent is a polysorbate surfactant selected from TWEEN®80 (2-[2-[3,4-bis(2-hydroxyethoxy)oxolan-2-yl]-2-(2-hydroxyethoxy)ethoxy]ethyl octadec-9-enoate) and TWEEN®20 (2-[2-[3,4-bis(2-hydroxyethoxy)oxolan-2-yl]-2-(2-hydroxyethoxy)ethoxy]ethyl dodecanoate).

15. The method of claim 11, wherein the chelating agent is selected from ethylenediamine tetraacetic acid (EDTA), ethylene glycol tetraacetic acid (EGTA), cyclohexanediamine tetraacetic acid (CDTA), hydroxyethyl ethylenediamine triacetic acid (HEDTA), diethylenetriamine pentaacetic acid (DTPA), dimercaptopropane sulfonic acid (DMPS), dimercaptosuccinic acid (DMSA), amino trimethylene phosphonic acid (ATPA), nitrilotriacetic acid (NTA), 1,3-propylene diamine tetraacetic acid (PDTA), Ethylene diamine disuccinic acid (EDDS), and ethylene glycol tetraacetic acid (EGTA) and acceptable salts thereof, and any combinations thereof.

16. The method of claim 15, wherein the EDTA salt is selected from diammonium EDTA, disodium EDTA, dipotassium EDTA, triammonium EDTA, trisodium EDTA, tripotassium EDTA, tetrasodium EDTA, tetrapotassium EDTA, calcium disodium EDTA, and combinations thereof.

17. The method of claim 11, wherein the formulation comprises: MSM at 5.4% w/w; di-sodium EDTA at 2.6% w/w; propylene glycol at 5.0% w/w, hydroxyethylcellulose (HEC) at 0.3%, TWEEN®80 (polysorbate 80) at 15.0% w/w, and optionally, guar gum or hydroxypropyl guar at 0.1% w/w.

18. The method of claim 11, wherein the age-related cataract is characterized by one or more of (a) loss of lens transparency caused by increased size and number of light-scattering particles and (b) loss of visual function.

19. The method of claim 11, wherein an improvement of visual function following administration of the formulation is determined by measuring one or both of (a) an increase in contrast sensitivity (CS) and (b) an increase in visual acuity (VA).

20. The method of claim 19, wherein an improvement of visual function comprises a clinically meaningful improvement (≥0.30 log CS) at ≥2 spatial frequencies in mesopic CS.

21. The method of claim 19, wherein an improvement of visual function following administration of the formulation is determined by measuring Average Lens Density.
